(19) United States
(12) Patent Application Publication
(10) Pub. No.: US 2006/0078541 A1
(43) Pub. Date: Apr. 13, 2006

(54) Method and formula for stem cells' stimulation, targeting release, trafficking and homing

(76) Inventor: Brian C. Giles, Montecito, CA (US)

Correspondence Address: SHEPPARD, MULLIN, RICHTER & HAMPTON LLP, 333 SOUTH HOPE STREET, 48TH FLOOR, LOS ANGELES, CA, 90071-1448, US

(21) Appl. No.: 11/241,196

(22) Filed: Sep. 30, 2005

Related U.S. Application Data
(60) Provisional application No. 60/615,197, filed on Oct. 1, 2004.

(51) Int. Cl. A61K 35/12; A61K 33/00

(52) U.S. Cl. 424/93.1; 424/722

(57) ABSTRACT

The present invention relates to the fields of pharmacology, regenerative medicine, electro-physiology, and stem cell therapy. In a method aspect, a method of stimulating stem cell release in a mammal is provided. The method includes the step of administering a composition to the mammal, and wherein the composition comprises from 500 ppm to 100,000 ppm of a cesium ion source, a rubidium ion source, or a mixture of the two.

### CROSS-REFERENCE TO RELATED APPLICATION

This application claims priority to U.S. Provisional Patent Application Ser. No. 60/615,197, filed Oct. 1, 2004 the entire disclosure of which is incorporated by reference.

### FIELD OF THE INVENTION

The present invention relates to the fields of pharmacology, regenerative medicine, electro-physiology, and stem cell therapy.

### BACKGROUND OF THE INVENTION

The use of embryonic stem cells (“ESC”s) for research purposes and for the development of treatments for a wide variety of diseases is the focus of substantial scientific and political interest. Significant socio-political opposition has arisen regarding ESCs due to ethical questions posed by cell harvesting methods and the implications of research involving human genetic manipulation.

Research and treatments involving adult stem cells provide an alternative to the dilemmas associated with ESCs. Much evidence has accumulated over the past few years indicating that adult bone marrow stem cells and other cell lineages potentially have pluripotent properties similar to ESC. This has led to the hypothesis that stimulation of in situ release of bone marrow cells might constitute a viable alternative to ESCs, in view of their use for the treatment and or prophylaxis of a wide variety of diseases.

Recently, it has been shown, that certain naturally occurring biomolecules have the ability to increase the number of circulating stem cells as well as their homing into tissues. The trafficking and homing of in situ released bone marrow cells is dependent on a variety signals that come from organs that need to be repaired in the context of normal cellular turnover and homeostasis. These mechanisms are mediated by apoptosis. Additionally, the immune system plays a major role in determining whether the stem cells find their target and are able to repopulate and regenerate the tissue involved. Of special importance are cytokines and other immunological response modifiers.

U.S. Pat. No. 6,784,155, for example, discusses stimulators of stem cell proliferation. Specifically, the patent reports methods for the isolation and use of peptide-based stem cell modulating factors for regulating the stem cell cycle and for accelerating the post-chemotherapy, peripheral blood cell recovery.

U.S. Pat. No. 6,841,147 presents further peptide-based stem cell factors that may be used to stimulate stem cell production. The factors and methods of peptide production are discussed. Furthermore, pharmaceutical compositions and method of treating disorders involving blood cells are reported.

U.S. Pat. No. 6,824,973 provides a method for promoting stem cell proliferation or survival by contacting a cell with a stem cell factor-like polypeptide. The polynucleotides encoding the polypeptides include nucleic acid sequences isolated from cDNA libraries from human testis cells, from human fetal skin, adult spleen, and human endothelial cells.

There is accordingly a need in the art for a non-peptide based treatment that induces the production, targeting and adhesion of adult stem cells in a subject. That is an object of the present invention.

### SUMMARY OF THE INVENTION

The present invention relates to the fields of pharmacology, regenerative medicine, electro-physiology, and stem cell therapy.

In a method aspect, a method of stimulating stem cell release in a mammal is provided. The method includes the step of administering a composition to the mammal, and wherein the composition comprises from 500 ppm to 100,000 ppm of a cesium ion source, a rubidium ion source, or a mixture of the two.

In another method aspect, a method of facilitating stem cell targeting in a mammal is provided. The method includes the step of administering a composition to the mammal, and wherein the composition comprises from 500 ppm to 100,000 ppm of a cesium ion source, a rubidium ion source, or a mixture of the two.

In another method aspect, a method of increasing stem cell adhesion to a target site is provided. The method includes the step of administering a composition to the mammal, and wherein the composition comprises from 500 ppm to 100,000 ppm of a cesium ion source, a rubidium ion source, or a mixture of the two.

In another method aspect, a method of increasing stem cell replication is provided. The method includes the step of administering a composition to the mammal, and wherein the composition comprises from 500 ppm to 100,000 ppm of a cesium ion source, a rubidium ion source, or a mixture of the two.

In another method aspect, a method of increasing stem cell propagation is provided. The method includes the step of administering a composition to the mammal, and wherein the composition comprises from 500 ppm to 100,000 ppm of a cesium ion source, a rubidium ion source, or a mixture of the two.

In another method aspect, a method of increasing stem cell homing is provided. The method includes the step of administering a composition to the mammal, and wherein the composition comprises from 500 ppm to 100,000 ppm of a cesium ion source, a rubidium ion source, or a mixture of the two.

In another method aspect, a method of increasing stem cell pluripotency is provided. The method includes the step of administering a composition to the mammal, and wherein the composition comprises from 500 ppm to 100,000 ppm of a cesium ion source, a rubidium ion source, or a mixture of the two.

In another method aspect, a method of improving stem cell traffiking is provided. The method includes the step of administering a composition to the mammal, and wherein the composition comprises from 500 ppm to 100,000 ppm of a cesium ion source, a rubidium ion source, or a mixture of the two.

In another method aspect, a method of improving stem cell genetic coding is provided. The method includes the step of administering a composition to the mammal, and wherein the composition comprises from 500 ppm to 100,000 ppm of a cesium ion source, a rubidium ion source, or a mixture of the two.

In another method aspect, a method of increasing stem cell survival rates is provided. The method includes the step of administering a composition to the mammal, and wherein the composition comprises from 500 ppm to 100,000 ppm of a cesium ion source, a rubidium ion source, or a mixture of the two.

In another method aspect, a method of increasing stem cell proliferation rates is provided. The method includes the step of administering a composition to the mammal, and wherein the composition comprises from 500 ppm to 100,000 ppm of a cesium ion source, a rubidium ion source, or a mixture of the two.

In a composition aspect, a composition is provided. The composition includes: from 500 ppm to 100,000 ppm of a cesium ion source, a rubidium ion source, or a mixture of the two; and, a polypeptide known to stimulate stem cell release.

In another composition aspect, a composition is provided that includes: from 500 ppm to 100,000 ppm of a cesium ion source, a rubidium ion source, or a mixture of the two; 1 Alpha protein; and either a glycol sugar or a polysaccharide.

### DETAILED DESCRIPTION OF THE INVENTION

Definitions

“pH” is the measurement of acidity and alkalinity that is measured by pH, which is defined as the negative logarithm of the Hydrogen ion activity: pH=−log(H+). The parameter pHe is the pH on the exterior and pHi is the pH on the interior of the cell.

A “free radical” is an atom or molecule with an unpaired electron. In other words, the electron is not paired with another electron in the formation of a chemical bond. Generally, this results in a molecule with a net magnetic moment, so it is paramagnetic. Free radicals are unstable and damage other chemicals, including DNA and RNA.

“Oxidative stress” refers to biochemical insult to a cell resulting from damaging reactions involving oxygen. Abundant oxygen is necessary to the energy metabolism of the cell, but the secondary reaction must be minimized and harmful chemicals detoxified.

“Effective amount” refers to an amount or quantity of the active substance that is sufficient to elicit the required or desired therapeutic response.

Introduction

Health can be influenced by many factors. One of the most fundamental factors is the correct function of homeostatic regulation of ionic concentrations, pH and membrane electrical potentials. Most biochemical activities of living cells are controlled by pH and ion concentrations; many key processes, such as energy metabolism and nerve and brain function, depend on membrane electrical potential. It is also known that ion movements, correct pH ranges and membrane electrical potential are closely linked through the action of ion specific gates, electrogenic carriers and other ion carriers in cell and organelle membranes.

Most diseases are caused by a reduction in cellular pH, pHe and pHi and progress though a complex series of pathological and electro-physiological changes from initial onset to full manifestation. Unfortunately, drugs currently on the market do not address the complexity of acidosis, hypoxia and inflammation disease processes and the underlying source, causes or mechanisms. This oftentimes makes them difficult to understand and treat without knowing the electro-chemical and electro-physiological mechanisms.

There are many drugs and therapies aimed at correcting specific disease symptoms that result from failure to maintain the optimum or near optimum pH range and the functional ionic physiological conditions. Preventative protection of the biological function from failure of homeostasis has generally been relegated largely to the realm of nutritional and lifestyle choices. The knowledge and understanding of ionic physiology enable us to provide formulations and methods that will make the protection of ionic homeostasis, such as minimizing the tissues and cells acidic and hypoxia damage.

The aberrant regulation of cellular homeostasis is a significant factor in the pathogenesis of disease onset. By elevating cellular pHe and cellular pHi to optimum or near optimum ranges reduces the excessive excitement of neurons, processes the stressful biological inflammatory complex free radical oxidative stress (such as super oxides, peroxides, oxyacids, alcohols and aldehydes), restores and stabilizes the ionic homeostasis, processes enzyme toxins and releases the useful molecular oxygen from its bound state. As an example, the central disorders of acute maladaptive reactions are oxygen deficit and acidity. The biochemistry of chronic and acute degenerative diseases reveals the same disorders as acute maladaptive reactions, which occur with a sub optimal pH environment that produces the acidic biological environment signaling pain.

Cesium and/or Rubidium ions provide an “electron bath.” The cells and tissues are bathed in electrons, and are then stabilized and no longer induce cellular tissue damage. The optimal electro-physiological environment (for stem cells) and normal healthy viable cells have a narrow and specific pHe ranging from 7.00 to 7.55, preferably between 7.30 and 7.45. The method and formula provides for tissue pH increase that obtains a reduction of the H+ ion migration and other fluxes, and provides electro-physiological resistance to degenerative processes and changes the ionic chemistry of the patient's cells and tissues including circulating stem cells.

### OBJECTS AND ADVANTAGES OF THE INVENTION

The present invention is unique in its approach to stem cell therapy, stimulating stem cell production by elevating (optimizing) the patient's systemic cellular pH, cellular pHe and cellular pHi and mitochondria pHe and pHi.

A further object of the invention optimizes the patient's biological environment on an exo-cellular (pHe) and intra-cellular (pHi) level; it provides a very wide therapeutic window (long-term effect).

A further advantage of the present invention is that it can be cost effectively administered as a stand alone therapy or as an effective adjunct in conjunction with other stem cell therapies and other bio-nutrients to stimulate release, targeting, adhesion, and replication of the stem cells.

A further object of the present invention is to employ a fundamentally unique approach in modifying the patient's internal electro-physical biological environment enabling stem cells stimulation for optimum stem cell homing, trafficking, targeting, adhesion and replication.

A further advantage of the present invention is that it provides in situ bone marrow stem cell stimulation and release effects with a wide therapeutic window.

A further object of the invention is to stimulate the metabolism and balance changes in the cellular ionic environment (mineral content). Mineral content and pH regulates stem cell shape change to obtain the optimum conditions for systemic cellular function reducing inflammatory response prior to, during and after stem cell therapy.

A further object of the invention is to obtain a cellular pHe above 7.30, preferably ranging from a pHe of 7.35 to 7.44.

A further object of the invention is to provide a pharmaceutical composition for ionic intervention that restores the critical fluids' ORP and electrolytes including 30 to 70 trace minerals and ions to prevent and restore the reduction of pH.

The invention discloses a method and formula for effectively reversing the aging of adult stem cells and restoring their volume shape (tensegrity) and restores their surface characteristics by modifying the electro-physical biological environment to optimal pHe and pHi values. This modification provides for full potential stem cell divisions, longevity, cell division, replication cycle extension and optimization of metabolic processes. The result is the extension of the patient's host cells, newly released stem cells and externally introduced stem cells.

There is a wide variety of other stem cell release sites, such as bone, muscle etc. Treating the patient with their own stem cells, i.e. (non-limiting example) by removing the patient's bone marrow, harvesting the stem cells therein, culturing them into a sufficient colony size and readministering the stem cells to the patient via injection, etc. Once implanted, the stem cells traveled to the injury site and intertwined with cells secreting SDF-1 alpha. The number of stem cells reaching the area of need correlates with the quantity of SDF-1 alpha that is secreted providing a composition for preparing a patient for externally introduced stem cell therapy. stem cells event counter and and encourages the promotion of cell membrane integrity, i.e., an increase in electrical gradient, an optimization of proton pathways, and promotion of healthy (electro-negative) cell membrane structure,

By administering the composition of the present invention to shift (increase) the systemic pH, stem cell and host stem cell's cellular pHe and cellular pHi, immune function is modulated. The immune system plays a prominent role in determining whether the stem cells from bone marrow find their target of need and are able to successfully populate and regenerate the tissue involved. Of special importance are cytokines and other immunological response modifiers involving the complete network of the immune system, including the brain.

The current invention provides methods and compositions for prevention and or treatment of diseases and disorders associated with dysregulation of cellular homeostasis, cell cycle regulation, regulating the balance between cell proliferation and apoptosis.

The composition may be administered with dosages until the targeted pH, pHe, and pHi ranges are obtained prior to stem cell release or the external introduction of stem cell therapy. Then dosages are adjusted to maintenence levels. When the patient saliva pH is elevated above 7.00 preferably between 7.20 to 7.45. The method employs a virtually non-toxic composition to electro-physically stimulate and release in situ bone marrow stem cells that are autologous and (matched) heterologous for replacing the cell population and balancing cell functions improving the ability of the stem cell surface characteristics to recognize the targeted area of inflammation, i.e. reduced pH. The composition systemically restores the electro-chemical environment reduces and eliminates stem cell rejection and improving chain migration optimizes electro-physiological environment and function.

The formula contains salts containing Cesium and or Rubidium ions to systematically neutralize and alkalinize the acidic biological environment and restore the optimum electrophysical environment. The composition's ionic activity is electro-chemical and electro-physical for obtaining enhanced therapeutic efficacy in stem cell growth release and replication cycles. Released adult stem cells in the optimum or near optimum pH ranges may be found to be as versatile as juvenile stem cells.

Cesium and rubidium potassium and magnesium ions provide for optimal cell pHi (intercellular) and optimal or near optimal pHe for regulating and optimizing mitochondrial function and the proton path. The ions are electro-physically selected and transported across cellular membranes by magnetic differential, tailoring the cesium and rubidium ions to the patient's cellular physiology such as, systemic pH, cellular pHe, cellular pHi electro-biological age, condition and bio-chemistry. By administering to a patient in need of such a therapy sufficient dosages to elevate the patient's pHe's ranging from 7.31 to 7.44, more preferably between 7.32 to 7.41 to optimize the targeting sites of injury and disease, clearing toxins from threatened tissue and replacing dead and dying cells. Stem cells follow, the signaling chemicals released during inflammation. From there, the cells fight inflammation, reduce scarring, release chemicals called growth factors that promote the health of remaining cells and signal multiply into replacement cells.. The stem cells fuse and convert to the right cell type.

The effects promoted by the composition of the present invention include those secondary to the inhibition of the large trans-membrane potassium movements resulting from acidosis energy metabolism. A sufficient dosage or dosages of the composition is administered to patients to obtain a targeted increase, systemically removing acidotic toxins and restoring the electrophysical environment by modulating to the optimum or near optimum pH ranges, Suitable for an adjunct with a variety of stem cell therapies. The composition may be combined with growth factors from inducing specific cell/tissue types. migrating to sites of need by electro-chemically sensing the reduced pH (inflammation) ranging from 5.20 to 7.25. correction of excessive sodium accumulation, cell and organ hydration, modification of membrane electrical potential, improvement of capacity of the ion exchange mechanisms, and optimization of bone marrow stem cell stimulation release, targeting, homing and trafficking and adhesion (fuse) with chain migration and replicate the specific cell lineage that is needed.

Delivery of Compositions

The composition of the present invention may be orally administered without previous dissolution, depending on dose, or prepared as an aqueous solution suitable for oral ingestion or formulated for IV injection. As an example, solutions for IV injection should be prepared with a chemical composition that renders the solutions pH acceptable—e.g., a sterile buffered saline solution isotonic to blood.

Additionally, suspensions of the active salt or salts compounds may be prepared as appropriate oily injection suspensions. A suitable lipophilic solvents or vehicles such as but not limited to, fatty oils such as sesame oil, or synthetic fatty esters, such as ethyl oleate or triglycerides, or liposomes, etc.

The compositions of the present invention may be administered by any acceptable route including, but not limited to, oral, periodic injections, intravenous infusion, rectal, vaginal, topical, nasogastric, transdermal application, sublingual, peritoneal, subcutaneous, sublingual, intramuscular, intrathecal, use of an implanted osmotic mini-pump or other slow-release methods or devices, etc. The composition described herein may be administered orally in a fluid medium, or in a powdered form such as tablets or capsules, etc.

Additionally, preparations that can be used orally include push-fit capsules made of gelatin, as well as soft, sealed capsules made of gelatin and a coating, such as manatol or sorbitol. Push fit capsules can contain active ingredients with electrolytes mixed with a filler or binders, such as cellulose lactose or starches, etc., with other nutrient additives. For oral pharmaceutical administration, the capsules are administered with sufficient alkaline water with a suitable complex low-glycemic index carbohydrate.

Compositions

Preferably, the compositions of the present invention include a cesium or rubidium ion source present in an amount sufficient to provide from 500 ppm to 100,000 ppm, preferably 1,000 ppm to 10,000 ppm, cesium and/or rubidium.

Any combination of cesium and/or rubidium salts which dissociate and ionize may be employed in the composition of the present invention, including, but not limited to: Arginate, Ascorbate, Aspartate, Caprylate, Chloride, Carbonate, Cysteinate, Citrate, Fumarate, Humic, Fulvate, Methionine, Glutamate, Gluconate, Glycinate, Asporotate, Lysinate, Succinate, Carbonate, Lactate, Malate, Tartrate, Sulfate, Phosphate, Nitrate, Flouride, Bromide, Iodide, Orotate, Asporotate, Bisulfonate, Lysinate, Fulvic, Succate, Carnate, Trisulfate, Lactobionate, Benzenesulfonate, Laurate, Benzoate, Bicarbonate, Benzoic, Caseinate, Bisufate, Mandelate, Bitartrate, Mesylate, Borate, Methylbromide, Methylnitrate, Calcium Edetate, Methylsulfate, Camsylate, Mucate, Napsylate, Clavulanate, N-Methylglucamine, Ammonium Salt, Dihydrochloride, Oleate, Edetate, Oxalate, Edisylate, Pamoate (Embonate), Esolate, Palmitate, Esylate, Pantothenate, Phosphate/Diphosphate, Gluceptate, Polygalacturonate, Salicylate, Stearate, Glycollylarsanilate, Hexylresorcinate, Subacetate, Hydrabamine, Hydrobromide, Tannate, Hydrochloride, Hydroxynaphthoate, Teoclate, Tosylate, Isothionate, Triethiodide, Panoate, Valerate, Acetate, Maleate, Malonate, Sulfate, and mixtures thereof. Or administering a combination of cesium and or rubidium salts

Additionally, other cesium and rubidium salts might be used in a wide variety of compositions, such as, but not limited to, various organic or metallic salts, if they meet the following requirements: (1) they must be pharmaceutically acceptable and have an acceptably low level of toxicity; (2) they must have sufficient high levels of cationic (alkaline) dissociation to allow the remaining negatively charged ions to effectively reduce cell and tissue acidity.

The salts included in the composition of the present invention may be formed using a wide variety of acids, including, but not limited to: hydrochloric, carbonic, humic, fulvic, sulfuric, acetic, lactic, tartaric, malic, succinic, etc. Malic acid, hydrochloric acid, carbonic acid and citric acid are preferred acids and mixtures thereof.

Potentiation of cesium and/or rubidium ionic action may be accomplished by inclusion of ingredients that enhance ionic pH physiology. Examples are electrolytes (saline compounds) such as potassium, sodium, and magnesium. Potassium, and other major electrolytes (e.g., sodium, calcium, chloride, bicarbonate, phosphate and sulfates) are added to the formulation in proportion to the potassium.

Other ingredients that may be included to potentiate the activity of cesium/rubidium ionic action include manganese, zinc, boron, glycol sugars, vitamin B2 (riboflavin), B12 (cyanocbalamin), and B6 (pyridoxine).

A solvent (e.g., water), especially in the case of an injectable solution, may be included in the composition. Where the solvent is water, a preferred method of manufacturing is to use water processed by means such as E.C.A. (electrolytic chemical activation) processing. The method produces aqueous solutions having certain characteristics. For example, an aqueous solution (water) for an oral treatment may be processed having a surface tension ranging from 30 to 73 dynes per cm², preferably ranging from 50 to 68 dynes per cm with an ORP (oxidative reduction potential) ranging between −20 m.v. to −350 m.v., preferably ranging between −50 m.v. to −300 m.v. (after dissociation). The pH for such compositions preferably ranges from 7.00 to 9.90, preferably between 8.00 to 9.10.

For compositions delivered orally, the following substances may be included, if they do not interfere with the dissociation and mobility of alkaline ions: flavorings, colorings, sweeteners, preservatives, and surfactants. Suitable surfactants are water-soluble organic compounds, and may be nonionic, cationic or amphoteric species throughout a suitable pH range.

Flavoring agents may be included in the oral composition if desired, as long as they do not substantially interfere with the ionic dissociation and mobility of cesium and/or rubidium. Such agents e.g., oils of citrus, lemon-lime, spearmint, menthol, cherry, wintergreen, sassafras and clove-are typically included to increase the palatability of a composition.

Flavoring agents may be lipophilic or hydrophilic. Lipophilic flavorants include, without limitation, wintergreen oil, oregano oil, bay leaf oil, peppermint oil, spearmint oil, clove oil, sage oil, sassafras oil, lemon oil, orange oil, anise oil, benzaldehyde, bitter almond oil, camphor, cedar leaf oil, marjoram oil, citronella oil, lavender oil, mustard oil, pine oil, pine needle oil, rosemary oil, thyme oil, cinnamon leaf oil, and mixtures thereof. Where used, lipophilic flavorants are typically included in the oral composition at a level from 0.01%-10% by weight, preferably from 0.05%-5.0% by weight, and more preferably from 0.1%-3.0% by weight.

Sweetening agents such as the following may also be included: D-tryptophan, dextrose, levulose, acesulfam, dihydrochalcones and sodium cyclamate. Such flavoring or sweetening agents are typically included in the composition in an amount from 0-5% by weight, preferably 0-2% by weight. Furthermore, coloring agents (e.g., colorants or pigments) may be added to improve the visual appearance of the composition. Suitable colorants include, without limitation, dyes such as FD & C blue No. 1, D & C yellow No. 10 and D & C yellow No. 3. A suitable and commonly used pigment is titanium dioxide, which provides a white color.

Other optional components for use in the present compositions include: antioxidants; vitamins (e.g., vitamin C and E); pH adjusting agents; and, plant extracts.

The composition of the present invention may optionally be combined with other stem cell therapies or compositions. Examples of such therapies and compositions are those discussed in U.S. Pat. Nos. 6,824,973, 6,784,155 and 6,841,147, the disclosures of which are incorporated-by-reference into this document for all purposes.

The efficacy of the formulation is monitored during therapy by clinical observations and diagnostic parameters particular to ionic therapy, including the monitoring of saliva pH, cellular pHe, cellular pHi and systemic pH. Observations and therapies related to the physiological stresses of Ionic pH therapy are included to prevent excessive therapy-related stress.

Chelation therapy may be included prior to stem cell therapy when necessary to reduce the levels of lead, mercury, cadmium, etc.

Bioceutics or neutraceutical therapy may be administered if required to promote stem cell release (increase the number of circulating stem cells) and simultaneously improve there homing to tissue sites of need.

In general, the dosage(s) required for stem cell therapy will be dependent on such factors as the patient's initial saliva pH, initial urine pH, venous pH, weight, age, diet, nutritional background, gender, physical symptoms and general health. Further factors include, duration and frequency of administration, chosen route of administration, reaction sensitivities, drug combination(s), and tolerance. The determination of an effective dose is well within the capability of those skilled in the art.

The retention pattern of cesium in adult humans may very widely with gender, age, and state of health with an increasing function of age throughout life. Cesium ions are nearly 100% assimilated, and their biological half life is 50 to 60 days.

A single high acute dose induces effects more or less similar to those induced by the chronic dose. Dosages should be administered with a meal and with extra clear water. Saliva pH indicates reaching the targeted pH range and the optimum electro-physical paramaters (targeted saliva pH ranges are from 7.00 to 7.50 with 7.20 to 7.40 being preferable). When the patient experiences numbness/parathesia on the center of the upper lip it is an indication of sufficient elevation of systemic pH to the optimum pH range and the optimum electro-physical parameters.

For oral administration, a loading dose should be provided, typically for 1 to 3 days. The administered dose for a 70 kg person is usually 500 mg to 1000 mg of a cesium salt per 24 hour period. Maintenance doses are then administered for a period of around 30 to 90 days, with about 60 being preferred. Again, for a 70 kg person, maintenance doses typically range from 100 mg to 500 mg cesium salt per 24 hours, with 200 mg to 300 mg being preferred.

For IV administration, a loading dose should be provided, typically for 1 to 3 days. The administered dose for a 70 kg person is usually 500 mg to 3000 mg of a cesium salt per 24 hour period. Maintenance doses are then administered as for the oral regimen described above.

Magnesium salts and potassium salts are typically administered along with the cesium salts in either the oral or IV methods. The magnesium and potassium salts are usually provided in a 1:1:1 weight ration with the cesium salt. In other words, the weight of administered cesium salt is approximately the same as that for the magnesium salt, which is approximately the same as that for the potassium salt.

Generally, administered doses of cesium salt are provided in a range from 2.5 mg/kg to 75 mg/kg. Pharmaceutical doses over 1 mEq per kilogram should only be used for critical (i.e., life threatening) cases with clinical monitoring for stress symptoms.

Dosage and administration are adjusted to provide sufficient levels of the active moiety or to maintain the desired effect. Long-acting pharmaceutical compositions may be administered every 24 hours or every other day, every week, or once every two weeks depending on half-life and clearance rate of the particular formulation.

Juvenile doses are generally about ½ of the adult range, depending on weight, etc. Salts containing ions of cesium or rubidium should not presently be administered to pregnant or lactating females or infants, or patients with arrhythmias without further studies or appropriate medical monitoring or intervention.

The patient's saliva, urine and blood pH should be monitored during stem cell therapy and the dosage appropriately adjusted. The goal of dosage adjustment is to partially or wholly restore and maintain the physiologic pH ranges including an optimum pHe ranging between 7.31 to 7.44 and maintaining the stem cells pHi above 6.40, preferably ranging from 6.50 to 6.80. Generally, the saliva pH reflects the ionic concentration and composition of the plasma; the saliva pH will gradually increase proportional to dosages.

Dietary Intervention

The method and composition are most effective if the patient's diet is nutritionally stimulating and does not contribute to acidotic stress. As an example, dietary foods and beverages with a pH below 2.5 should be completely eliminated. Preferably low glycemic index foods are administered during therapy. Oral fluid uptake should be derived primarily from water with a pH ranging from 7.00 to 9.50, preferably gradually increasing the pH range of the fluids consumed from 7.00 to 9.40, more preferably between 9.00 to 9.30. Any foods with pH below 2.50 and beverages with low pH results from mineral acids such as phosphoric acid should be eliminated. A neutral or slightly alkaline diet is preferably administered to the patient during the stem cell therapy.

Cesium and rubidium ions used in the present are separate and distinct from man-made isotopes of cesium and rubidium.

### EXAMPLES

The examples below are provided to illustrate the subject invention and are not included for the purpose of limiting the scope of the invention.

### Example 1

### Oral Aqueous Solution for Stem Cell Therapy

The following is are exemplary amounts of various ingredients of composition of the present invention, as provided to a subject over a 24 hour period:
a. cesium carbonate, rubidium carbonate of mixtures therof ranging from 500 mg to 5,000 mg, preferably 2,000 mg to 5,000 mg;
b. potassium (preferably as phosphate, gluconate and/or acetate) 100-500 mg;
c. calcium 500 to 2,500 mg;
d. magnesium citrate 200-1500 mg;
e. manganese (citrate or orotate) 1 to 20 mg;
f. iodine 10-40 mcg.;
g. selenium (Selenomethionine) 5-20 mcg;
h. vanadium (vanadyl sulfate) 1-5 mg;
i. zinc (gluconate/asporotate) 3-20 mg;
j. Vitamin D3 (cholecalciferol) 1,000 to 4,000 IU;
k. Vitamin A 1,000 to 3,000 IU;
l. Vitamin C (buffered L-ascorbic acid) 500 to 1,500 mg preferably time released;
m. malic acid 5-25 mg;
n. Coenzyme Q 10 (ubiquinone) 10-30 mg;
o. B3 (methyl nicotinate) 20-30 mg;
p. B6 25-100 mg;
q. B12 (cyanocobalmin) 10-100 mcg.

1. A method of stimulating stem cell release in a mammal, wherein the method comprises administering a composition to the mammal, and wherein the composition comprises from 500 ppm to 100,000 ppm of a cesium ion source, a rubidium ion source, or a mixture of the two.

2. The method according to claim 1, wherein the composition comprises from 500 ppm to 100,000 ppm of a cesium ion source.

3. The method according to claim 1, wherein the composition comprises water having a surface tension ranging from 30 to 73 dynes per cm².

4. The method according to claim 1, wherein the composition comprises from 1,000 ppm to 10,000 ppm of a cesium ion source, a rubidium ion source, or a mixture of the two.

5. The method according to claim 1, wherein the composition is in the form of a capsule or tablet.

6. A method of facilitating stem cell targeting in a mammal, wherein the method comprises topically administering a composition to the mammal, and wherein the composition comprises from 500 ppm to 100,000 ppm of a cesium ion source, a rubidium ion source, or a mixture of the two.

7. The method according to claim 6, wherein the composition comprises from 500 ppm to 100,000 ppm of a cesium ion source.

8. The method according to claim 6, wherein the composition comprises water having a surface tension ranging between 30 and 73 dynes per cm².

9. The method according to claim 6, wherein the composition comprises from 1,000 ppm to 10,000 ppm of a cesium ion source, a rubidium ion source, or a mixture of the two.

10. The method according to claim 6, wherein the composition is in the form of a capsule or tablet.

11. A composition comprising:
a) from 500 ppm to 100,000 ppm of a cesium ion source, a rubidium ion source, or a mixture of the two; and,
b) a polypeptide known to stimulate stem cell release.

12. The composition according to claim 11, wherein the composition is in the form of an aqueous solution or suspension.

13. The composition according to claim 11, wherein the composition is in the form of an oily suspension.

14. The composition according to claim 11, wherein the composition comprises cesium citrate.
